(19) United States
(12) Patent Application Publication
(10) Pub. No.: US 2004/0073124 A1
(43) Pub. Date: Apr. 15, 2004

(54) Method of using a matched filter for detecting QRS complex from a patient undergoing magnetic resonance imaging

(75) Inventor: Leon Axel, Philadelphia, PA (US)

Correspondence Address: DARBY & DARBY P.C., Post Office Box 5257, New York, NY, 10150-5257, US

(73) Assignee: New York University

(21) Appl. No.: 10/627,367

(22) Filed: Jul. 24, 2003

Related U.S. Application Data
(60) Provisional application No. 60/398,174, filed on Jul. 24, 2002.

(51) Int. Cl. A61B 5/04

(52) U.S. Cl. 600/509

(57) ABSTRACT

The invention relates to methods and systems for detecting QRS complex in a ECG of a patient undergoing magnetic resonance imaging and automating data acquisition in the magnetic resonance imaging machine upon detection. The method and system operates by recording an ECG signal sample from the patient, receiving a real-time ECG signal from a patient undergoing MRI and correlating the real-time ECG signal with a previously determined QRS complex template derived from the ECG signal received from the patient before undergoing MRI.

### BACKGROUND OF THE INVENTION

1. Field of the Invention

The present invention relates generally to a method for the detection of the initiation of a heartbeat as a patient undergoes magnetic resonance imaging, and relates particularly to the use of a matched filter that synchronizes the initiation of the readings of the magnetic resonance imaging machine with the readings of the detected QRS complex in the matched filter.

2. Description of the Related Art

The term “electrocardiogram” is defined as a test that measures the electrical activity of the heart. An electrocardiogram measures the rate and regularity of the heartbeat as well as the size and position of the chambers, any damage to the heart, and the effects of any drugs or devices that regulate the heart. Through the use of an electrocardiogram, various actions of the heart can be recorded.

The term “cardiac cycle” comprises atrial depolarization, ventricular depolarization, and ventricular re-polarization. The heart comprises at least two atria and two ventricles. The cardiac cycle involves the left atrium and left ventricle taking oxygenated blood from the pulmonary system and pumping it into the rest of the body and the right atrium and right ventricle taking deoxygenated blood from the body and pumping it to the lungs. The first step in the cardiac cycle comprises atrial depolarization. During atrial depolarization, the atrium contracts, pushing blood into the ventricle to fill it. The second step in the cardiac cycle comprises ventricular depolarization. After ventricular depolarization, the ventricle contracts, pushing blood into the aorta. The final step in the cardiac cycle comprises ventricular re-polarization. After ventricular depolarization, the ventricle relaxes and refills with blood. By means of an electrocardiogram, each step in the cardiac cycle can be recorded.

The term “P wave” depicts the atrial depolarization step of the cardiac cycle in an electrocardiogram.

The term “QRS complex” depicts the ventricular depolarization step of the cardiac cycle in an electrocardiogram. QRS complex is in fact a set of three waves namely the Q-, R-, and S-waves. QRS complex is generally associated with the initiation of the heartbeat.

The term “T wave” depicts the ventricular re-polarization step in the cardiac cycle in an electrocardiogram. It should be noted that the sounds made by the heart in the cardiac cycle are observed at the S and T waves.

The term “magnetic resonance imaging” is defined as a test that uses magnets and radio waves to construct images of the body. Magnetic resonance imaging is based on the magnetic properties of atoms. A magnetic resonance imaging machine produces a magnetic field approximately 10,000 times stronger than the earth's magnetism. Hydrogen atoms within the body align with the magnetic resonance imaging machine's magnetic field. Radio frequency magnetic fields, close in frequency to those of ordinary FM radio stations, are then broadcast towards the aligned hydrogen atoms. The aligned hydrogen atoms will then return a signal to the magnetic resonance imaging machine, which is used to reconstruct an image reflecting the local strength of the signal. The subtle differences of hydrogen atom alignment from one body tissue to another enable the magnetic resonance imaging to differentiate organs and potentially contrast tissue differences, such as between benign and malignant tissues.

Detection of QRS complex in the electrocardiogram (ECG) is essential for the synchronization of the cardiac cycle with cardiac imaging used in magnetic resonance imaging (“MRI”) devices. Past QRS complex detection techniques for a patient undergoing magnetic resonance imaging have been frequently unacceptable. First, detection of the QRS complex in the ECG while a patient underwent MRI was attendant with delays. Oftentimes, patients undergoing an MRI examination would wait while the technician rearranged the ECG test probes through a “trial and error” approach until a clear ECG signal was received. Once the ECG test probes were appropriately arranged, the technician would set the MRI machine to initiate MRI data acquisition upon the detection of the QRS complex. In so doing, the technician would enable the synchronization of the initiation of the heartbeat cycle with MRI data acquisition.

Many conditions complicate a technician's ability to find the clearest ECG signal. First, the magnet of the MRI machine complicates a technician's ability to find the clearest ECG signal. The strong magnetic field in the MRI magnet causes voltages known as flow artifacts to be induced by movement of the patient's blood. Just as QRS complex in the ECG signal has its own voltage potential, so too does moving blood. Consequently, the voltage potentials of the moving blood often make the voltage potentials of the QRS complex in the ECG signal indistinguishable from the voltage potentials associated with interference from flow artifacts. Second, each patient has a unique ECG signal with a corresponding unique QRS complex which further complicates a technician's ability to find the clearest ECG signal. Some patients have a weak heart and consequently, a weak ECG signal emanates from the ECG machine for such patients. Sometimes a patient's heart is so weak, that no arrangement of ECG probes will make QRS complex detection possible. By way of example, for a patient who has had a heart attack, scar tissue exists in the heart. Such scar tissue inhibits the patient's heart beat generally, and in particular, the strength of the ECG signal. Other such conditions that can inhibit an individual patient's ECG signal include the existence of fluid around the heart (commonly known as a pericardial effusion), and overinflated lungs such as caused by emphysema.

In sum, successful detection of the QRS complex in an ECG signal can be frustrated by several factors. Once the hurdle of obtaining a clear ECG signal is overcome, the next hurdle faced by the technician involves identifying the QRS complex in the ECG signal. Several techniques are available to the technician to identify the QRS complex so as to synchronize the MRI machine with the initiation of the heartbeat, however most are frequently unacceptable. Some such techniques include: (1) identifying QRS complex in the ECG signal through the use of a voltage detector; (2) measuring of the slope of a central portion of the QRS complex approach in the ECG signal; (3) identifying the timing sequence of QRS complex in the ECG signal; (4) physically restraining the patient; (5) the wavelet analysis approach; and (6) the vectorcardiogram approach which will be discussed in turn below.

First, the voltage detector approach will be discussed. Generally speaking, in the absence of interference, QRS complex will have the highest voltage value in the ECG signal. The voltage detector approach capitalizes on this principle. Consequently, if a voltage detector detects a voltage above a predetermined threshold, generally speaking, such voltage should correspond to QRS complex. Under the voltage detector approach, MRI readings are then triggered upon detection of any voltage above the predetermined threshold.

The voltage detector approach is a simple amplitude thresholding technique which is easy to implement. That being said, because the voltage associated with QRS complex deviates from the remaining components of the ECG signal by just millivolts, often the voltage detector approach will falsely trigger MRI readings based upon interference and not QRS complex. Thus, in order for the voltage detector approach to properly initiate MRI upon QRS complex detection, the interference patterns in the ECG signal must never exceed the millivolt voltage deviation associated with QRS complex which is unrealistic.

A second prior art attempt to identify QRS complex in an ECG signal and thereupon initiate MRI readings involves measuring the slope of a central portion of the QRS complex and comparing the measured slope with a preset range of values indicative of QRS complex slope. Once the comparison indicates a correlation, MRI readings are initiated. As with the voltage detector technique, however, this technique is flawed because interference patterns could render it impossible to find QRS complex in the ECG signal, let alone, measure the slope of the QRS complex.

A third prior art approach, namely the timing sequence approach, involves filtering interference from QRS complex and associating a time series during which QRS complex appears in the ECG signal. The time series is then sent to the MRI, which then initiates MRI readings in accordance with the time series. However, this prior art technique is flawed because automatically correlating MRI readings with a time series does not compensate for times when QRS complex occurs prematurely or belatedly in the real-time ECG signal while the patient undergoes MRI.

A fourth prior art approach minimizes interference in the ECG signal by restraining patient movement. Patient movement causes not only interference patterns in the ECG signal, but also blurs the images created by the magnetic resonance imaging. By way of example, a patient may be asked to hold their breath for a prescribed period of time while the patient undergoes MRI. Besides requiring the patient to act unnaturally, this technique is deficient because at times it is important for QRS complex to be evaluated as a function of the movement of a patient, for example, as a function of breathing.

A fifth prior art approach, namely the wavelet analysis approach, involves analyzing the ECG signal with a set of different scaled versions of a suitable wavelet basis function. QRS complex generally depicts a consistent peak across a range of scales. While the wavelet analysis more reliably detects QRS, wavelet analysis is relatively slow in execution and consequently, often the early phase of cardiac contraction cannot be captured by MRI.

A sixth prior art approach, also known as the vectorcardiogram approach, involves: (1) acquiring a sample ECG signal reading while the patient is outside the MRI; (2) determining the vector combination of the ECG voltage channels corresponding to QRS complex, also known as “the voltage vector space;” and (3) monitoring the ECG signal for the voltage vector space while the patient is inside the MRI. The vectorcardiogram approach is computationally complex. Another problem with the vectorcardiogram approach is that in order for the vectorcardiogram approach to discriminate QRS complex from interference, the vectorcardiogram approach assumes that interference voltages differ significantly from the QRS voltage vector space. However, interference voltages may be in the same region of the voltage vector space as the QRS complexes, and consequently the vectorcardiogram approach may also result in false MRI triggering.

It is thereby desirable to design a QRS complex detection method for implementation in MRI, which is computationally simple and consequently, results in minimal MRI trigger delay, and which is minimally affected by interfering voltages. In addition, what is needed is a QRS complex detection method for implementation in MRI which does not rigidly rely on a specific time series or voltage level associated with QRS complex in the ECG signal for MRI triggering. Furthermore, a QRS complex detection method is desirable which minimizes patient inconvenience, such as a method that eliminates separate testing of the ECG signal inside the MRI versus outside of the MRI and/or a method that eliminates the requirement of asking the patient to hold their breath. The present invention solves these aforementioned problems as well as others.

It should be noted that the references cited and discussed in the description of this invention are provided merely to clarify the description of the present invention. The recitation and/or discussion of these references is not an admission that any such reference is “prior art” to the invention described herein. All references cited and discussed in this specification are incorporated herein by reference in their entirety and to the same extent as if each reference was individually incorporated by reference.

### SUMMARY OF THE INVENTION

The present invention is directed to a system and method for automating an initiation of MRI data acquisition upon correlation of a real-time ECG signal of a patient undergoing MRI with a predescribed template. The method includes the step of correlating a predescribed template with a continuous-in-time ECG signal of a patient. The predescribed template is representative of a time course unique to a subsection of the ECG signal for the patient in a series of subsections of the ECG signal for the patient. The method also includes the step of determining a threshold that when exceeded indicates that the continuous-in-time ECG signal substantially correlates with the predescribed template. Finally, the method includes the step of correlating a real-time ECG signal of the patient with the predescribed template while the patient undergoes MRI.

### BRIEF DESCRIPTION OF THE ILLUSTRATED EMBODIMENTS

The foregoing and other features of the present invention will be more readily apparent from the detailed description in drawings of illustrative embodiments of the present invention wherein like reference numbers refer to similar elements throughout the several views and in which;

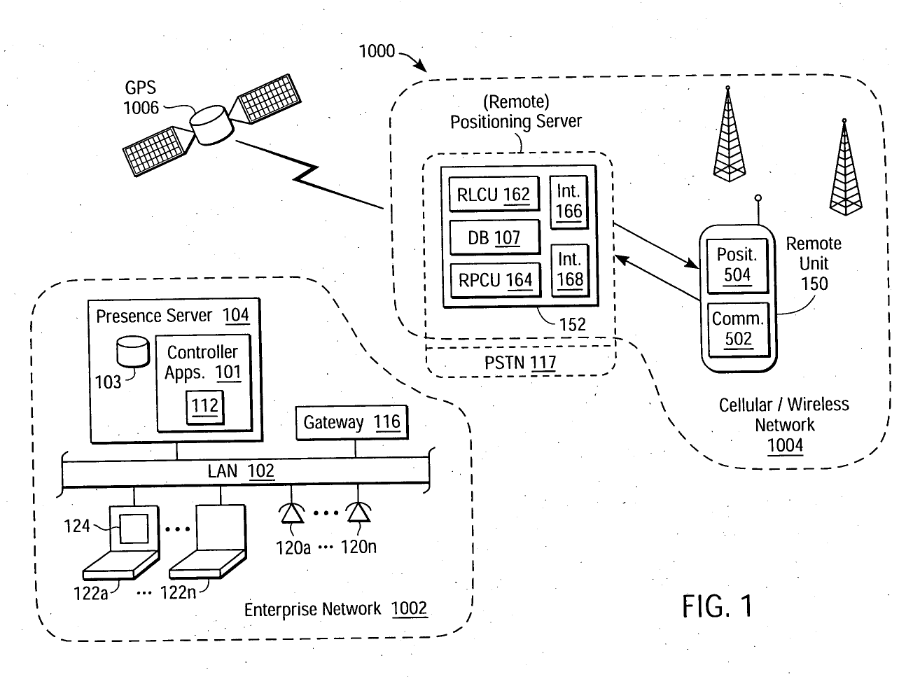

FIG. 1 is a system overview of the present invention in accordance with the first and second illustrated embodiments;

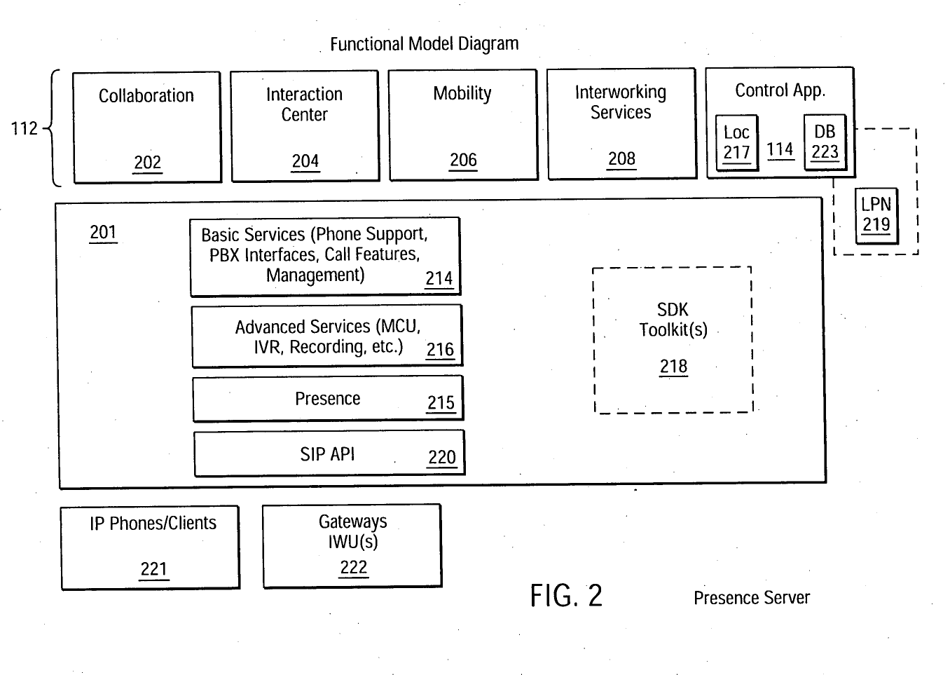

FIG. 2 is a flow diagram illustrating the steps of the present invention in accordance with a first illustrated embodiment;

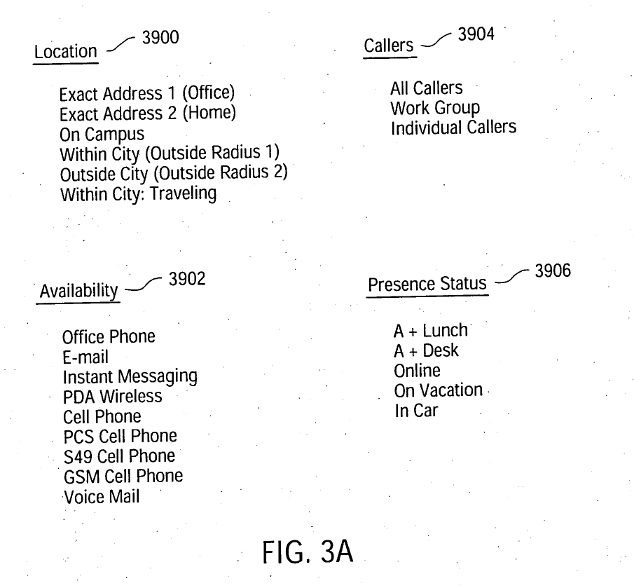

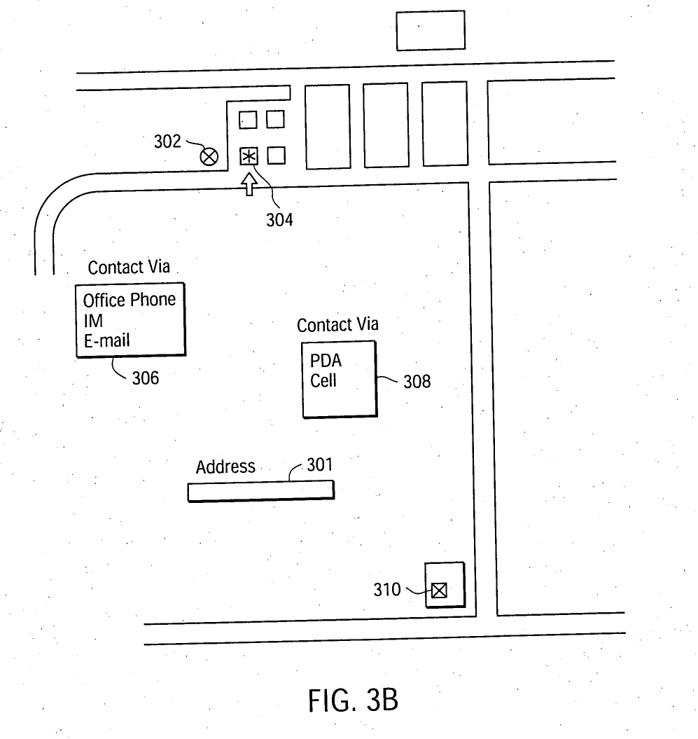

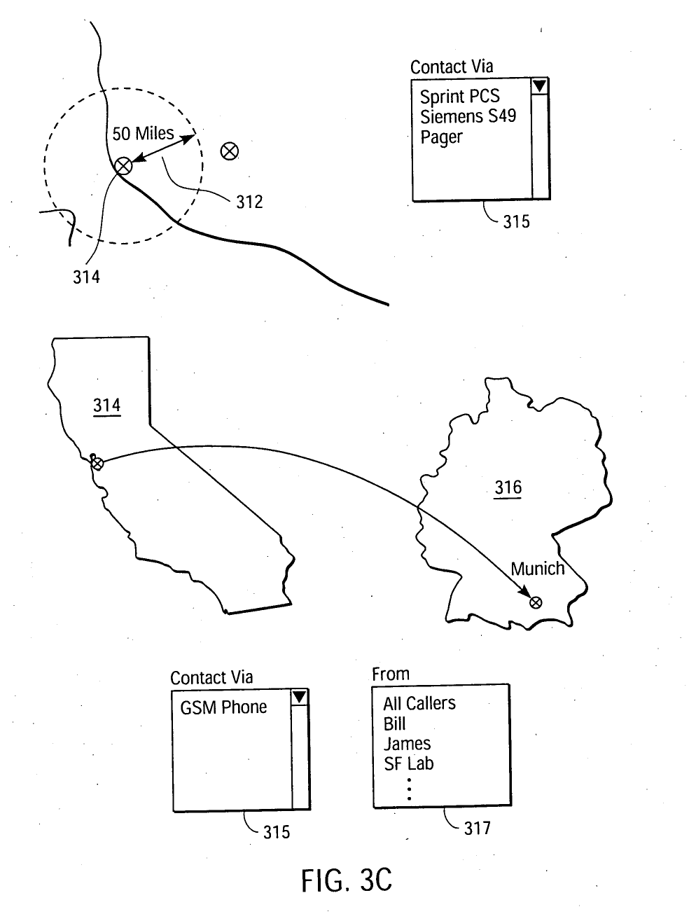

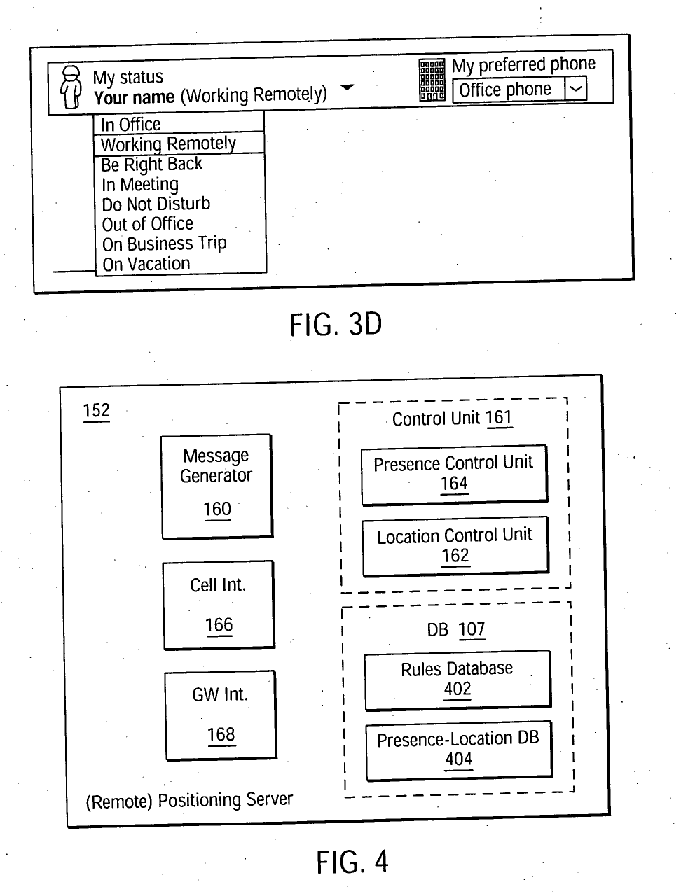

FIG. 3 is a flow diagram illustrating the steps of the present invention in accordance with a second illustrated embodiment;

FIG. 4 is a representative QRS complex as it would appear in an ECG signal;

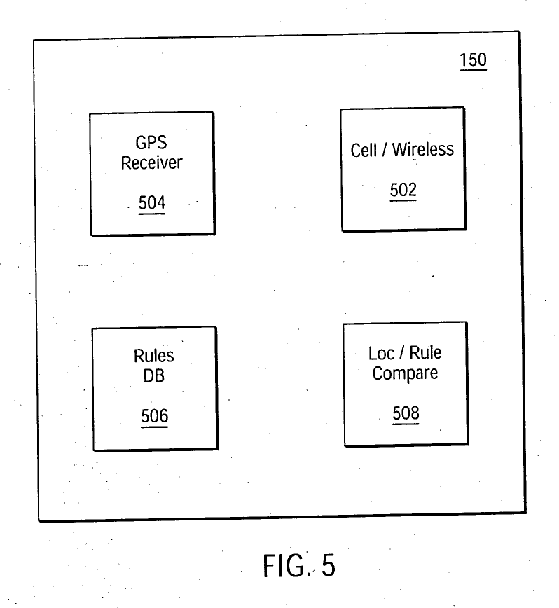

FIG. 5 depicts the two stages of the correlation filtering process;

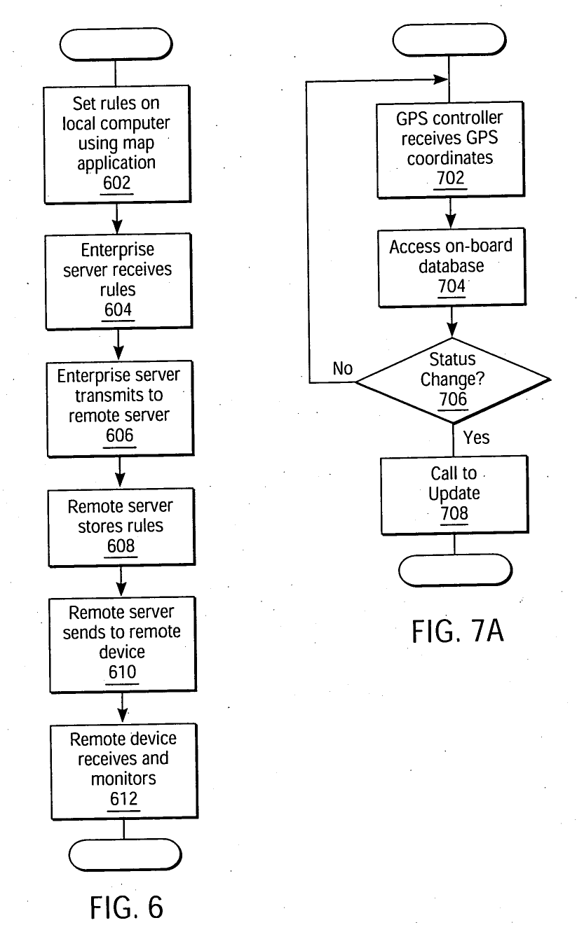

FIG. 6 depicts the QRS template generation process in accordance with the present invention;

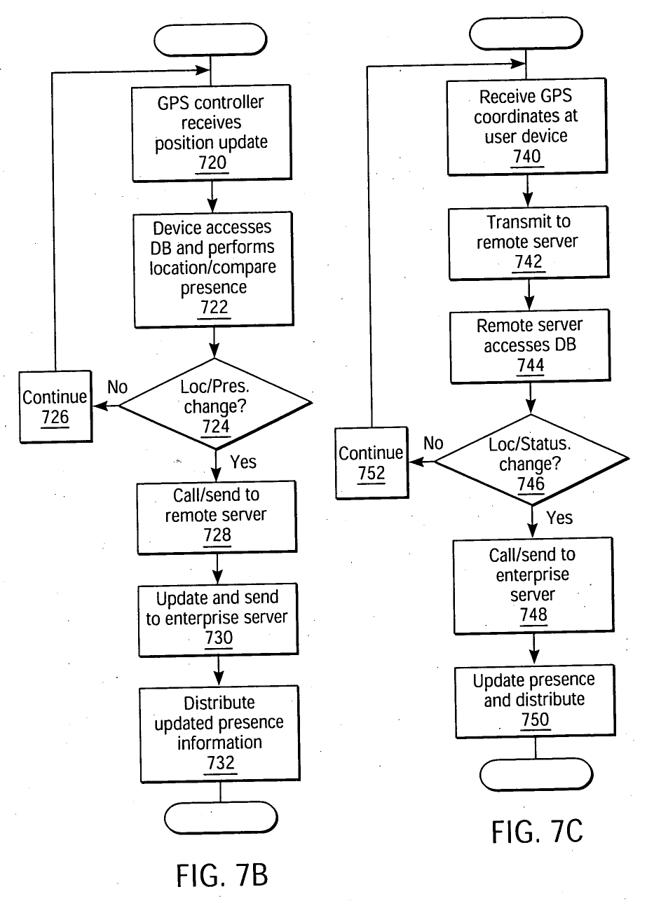

FIG. 7 depicts the multichannel correlation function process in accordance with the present invention which is expressed mathematically by equations (1-3);

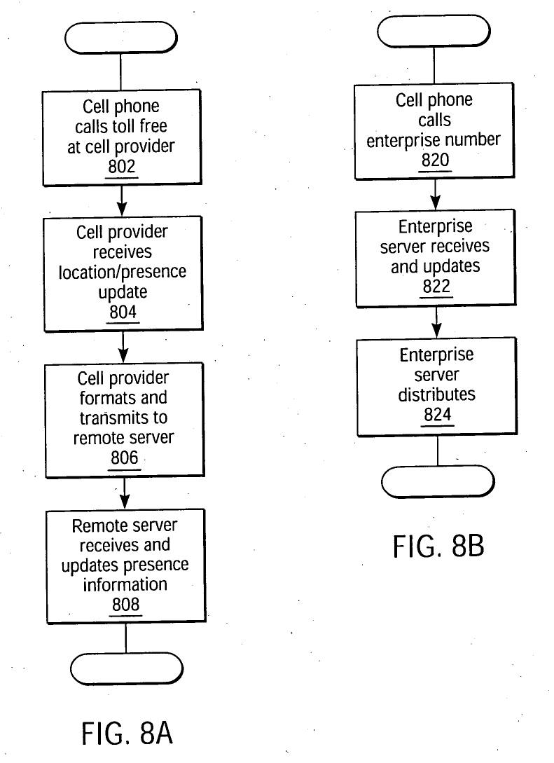

FIGS. 8A, 8B depict the ECG signals recorded (a) outside the MRI magnet for each of the three channels and (b) inside the MRI magnet for each of the three channels, respectively;

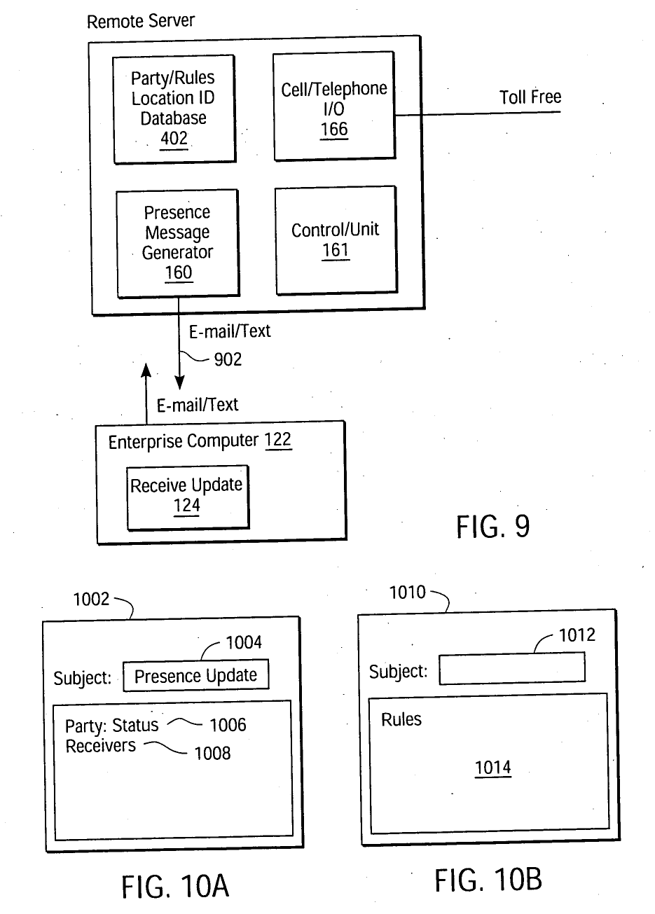

FIG. 9 depicts the QRS templates generated for each channel shown in FIG. 8B;

FIG. 10 depicts the correlation of each channel shown in FIG. 8B with its corresponding template generated in FIG. 9;

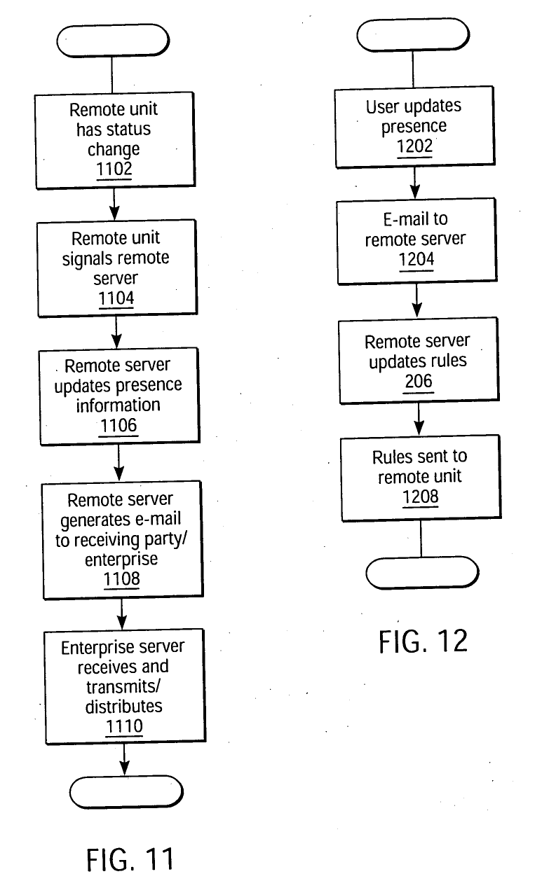

FIG. 11 depicts the net correlation of the three channels in FIG. 10 weighted in accordance with equations (2-3), along with the corresponding original ECG signals shown in FIG. 8B, and the triggers generated upon reaching the threshold; and,

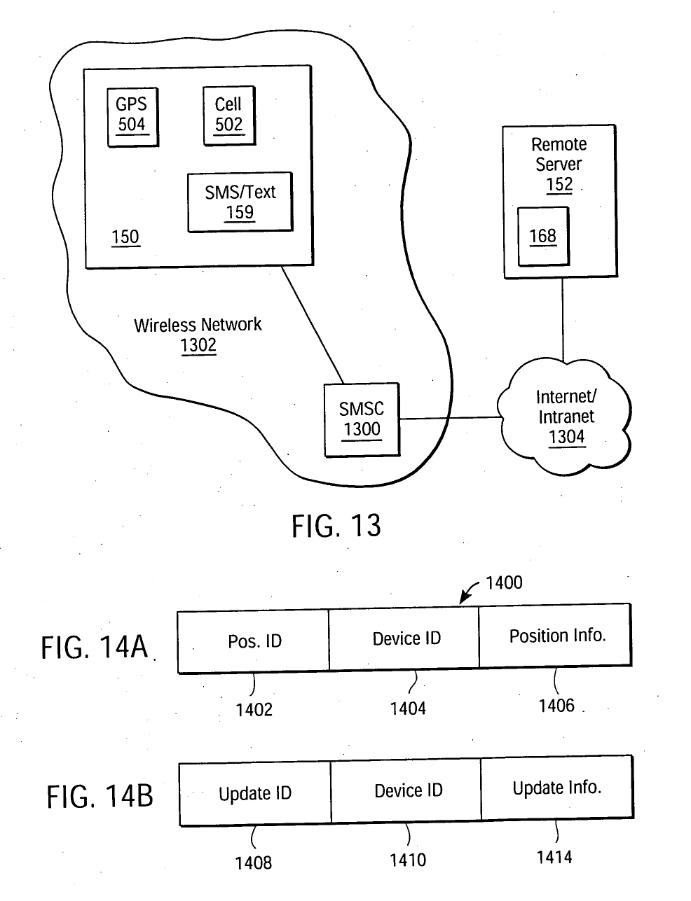

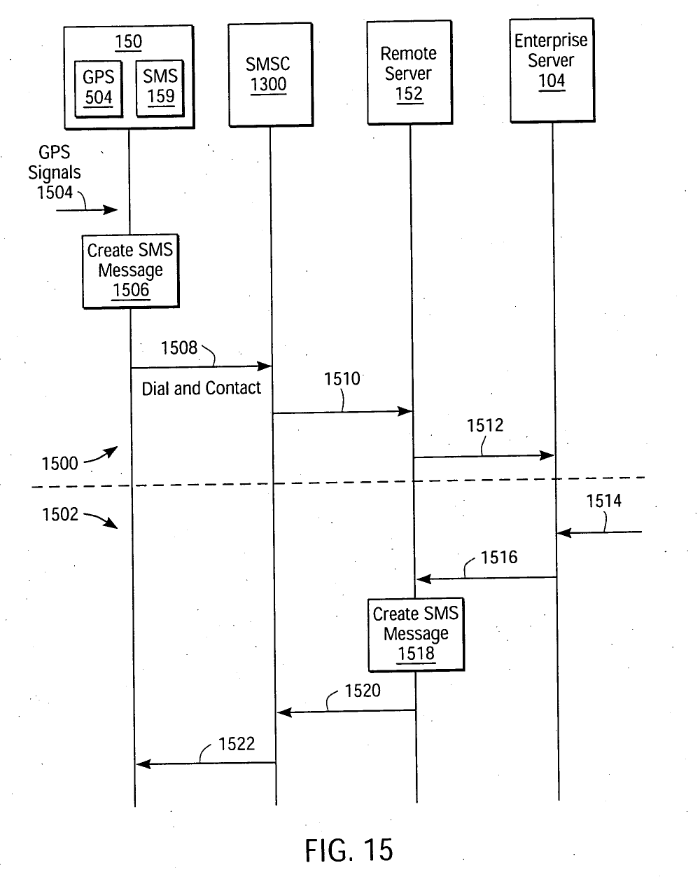

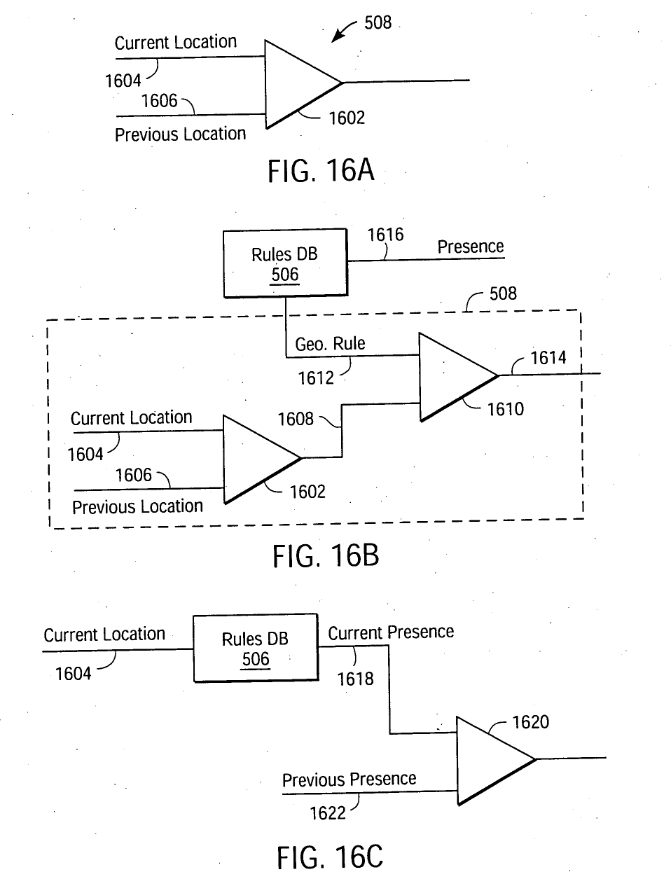

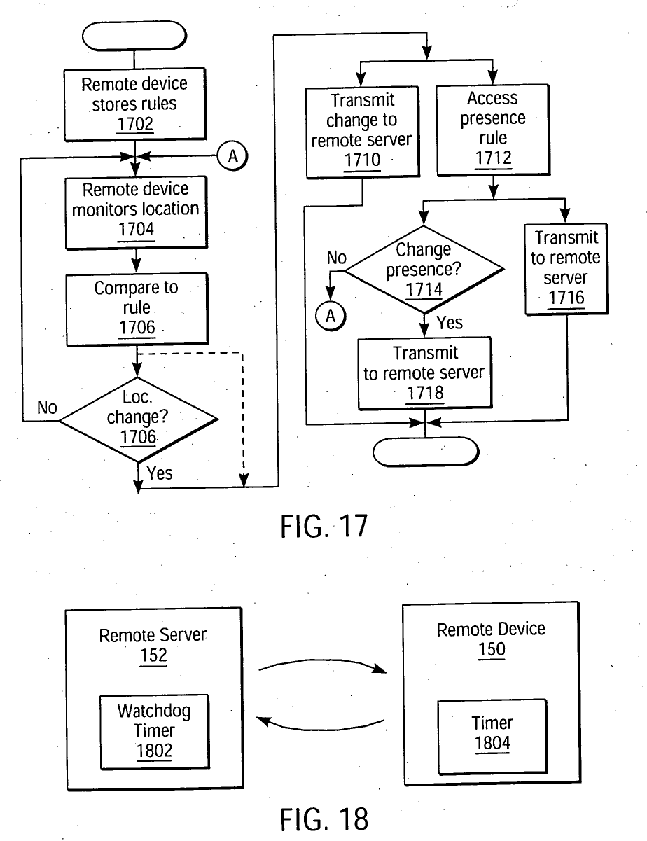

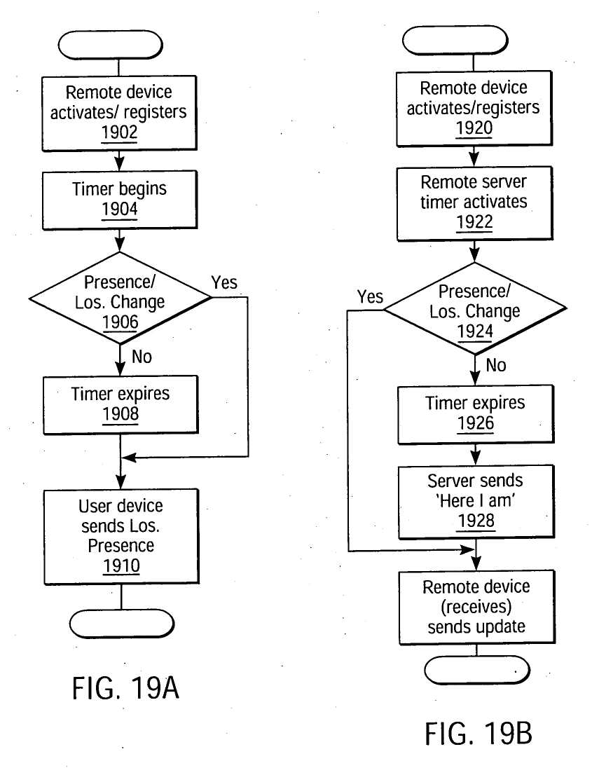

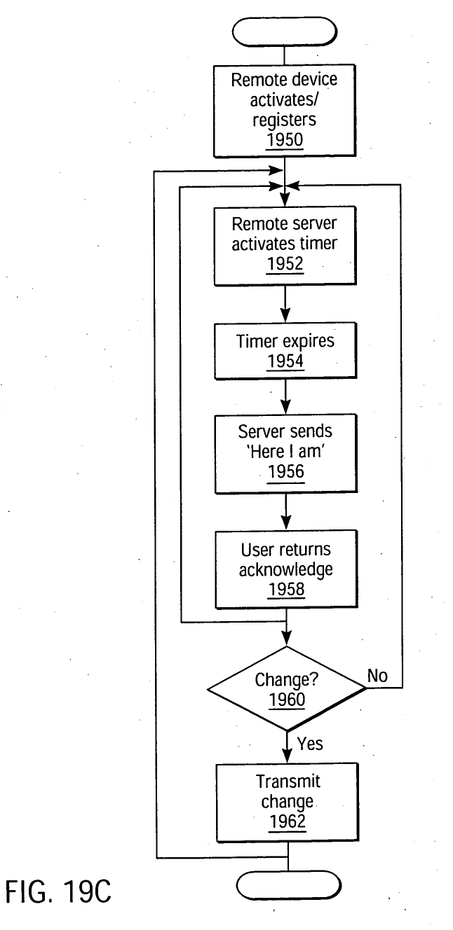

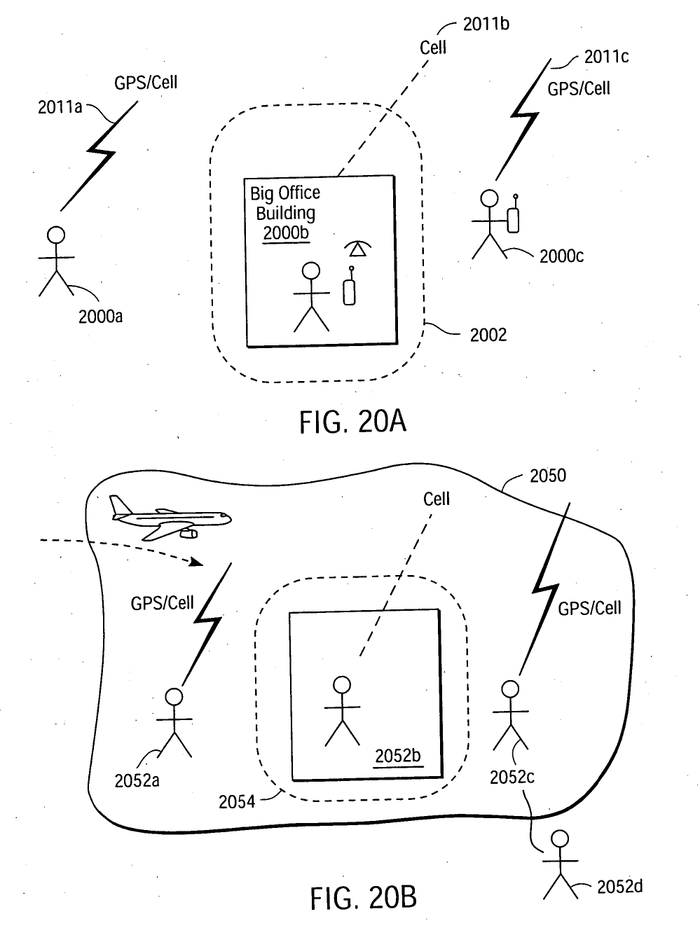

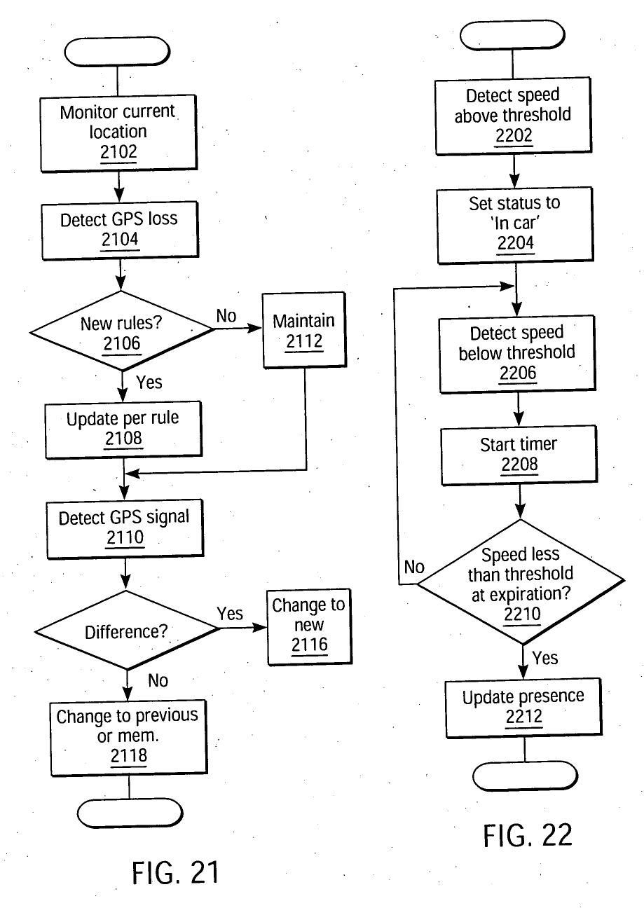

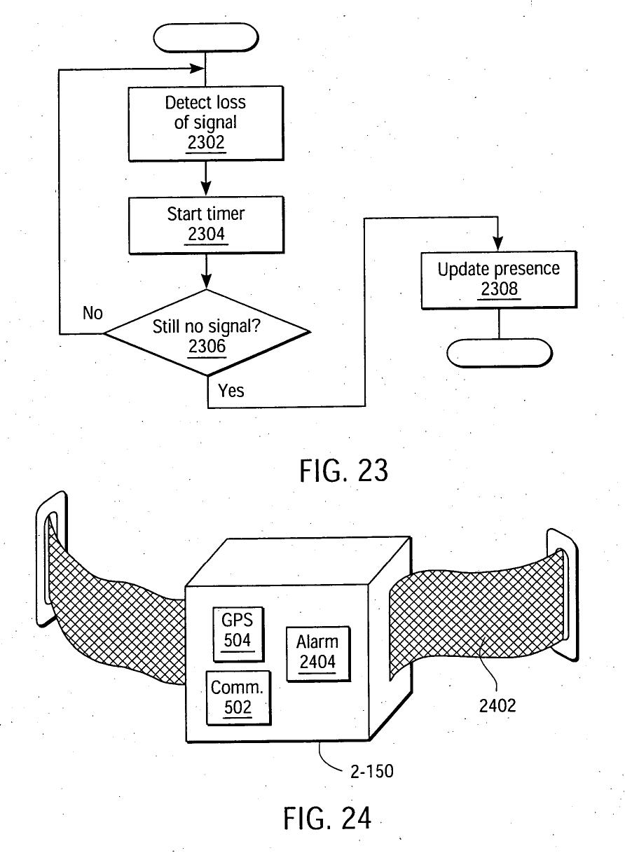

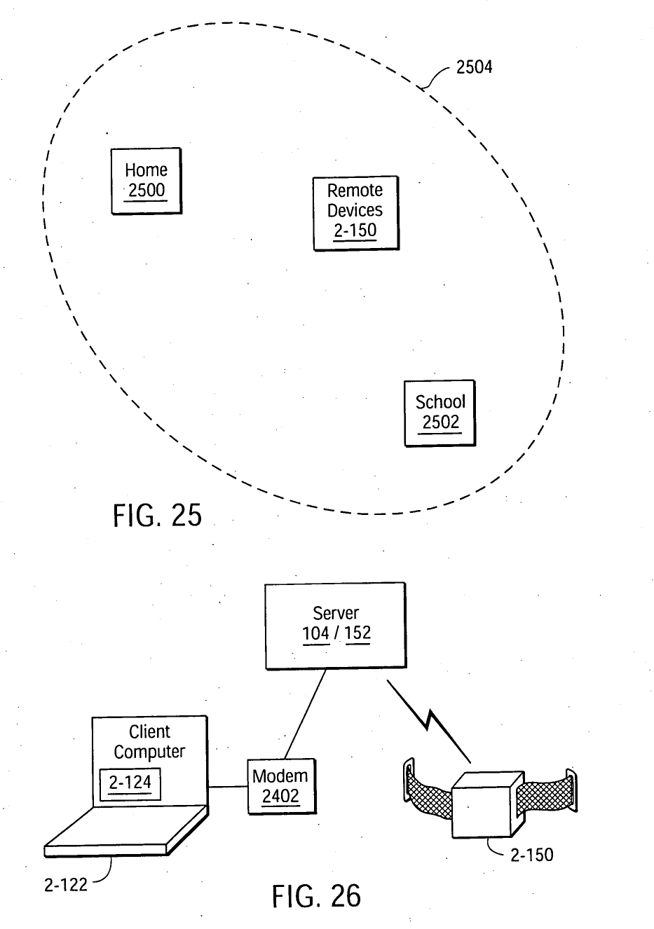

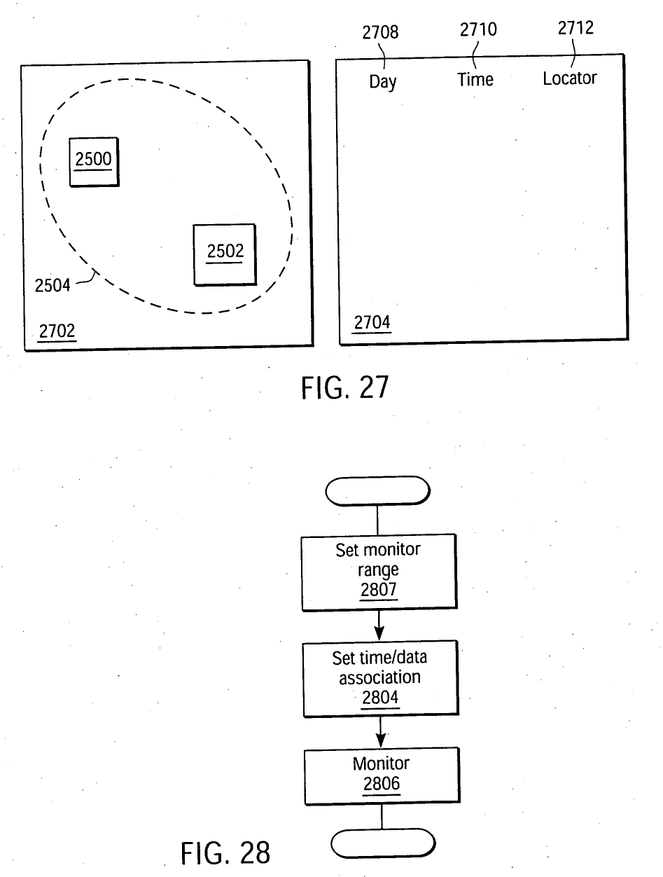

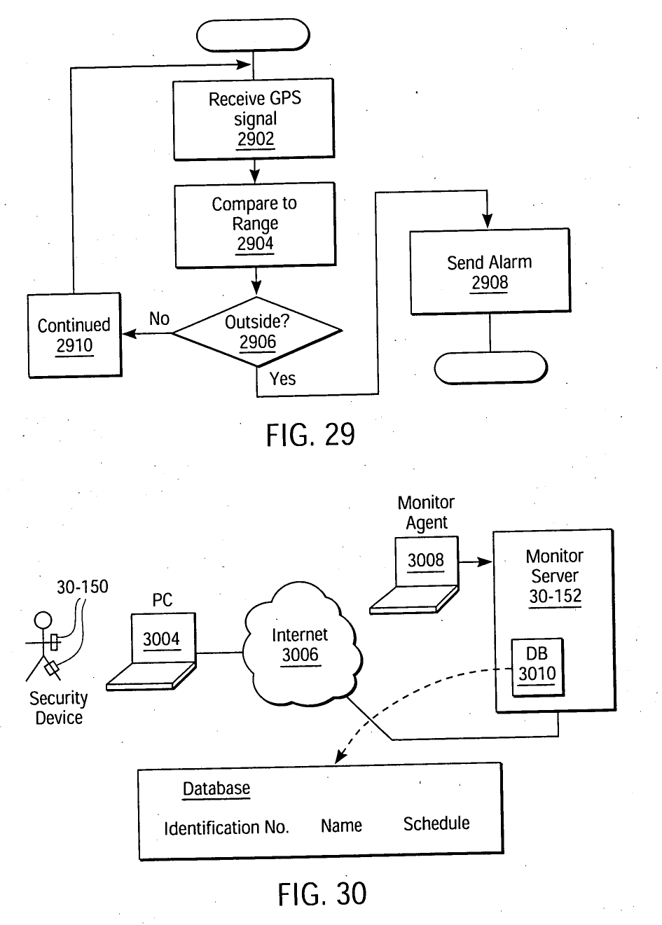

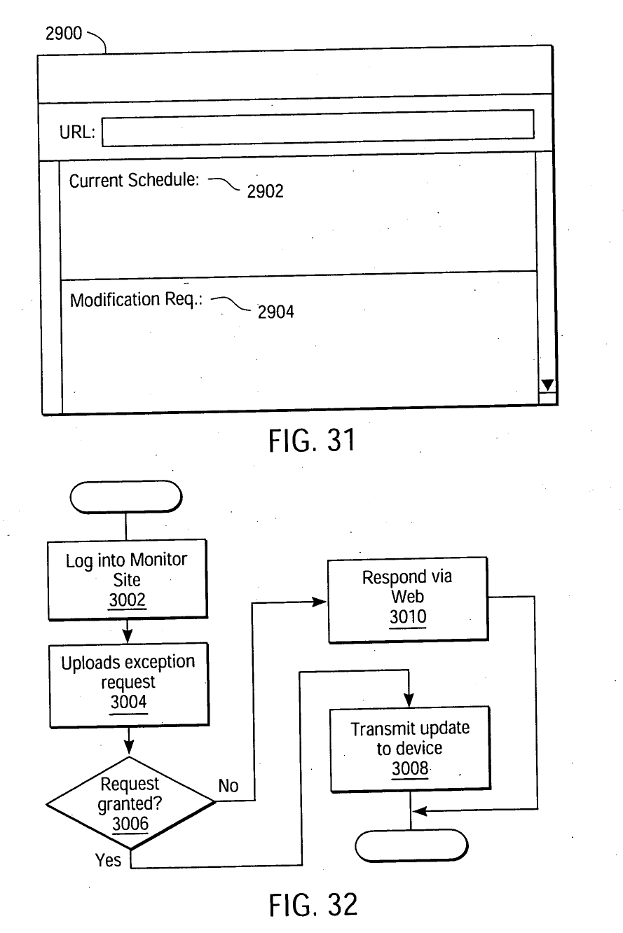

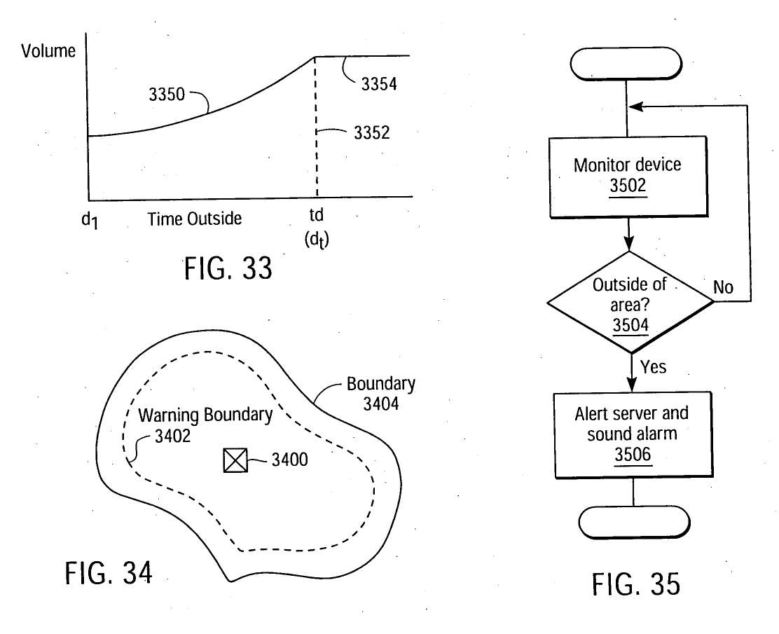

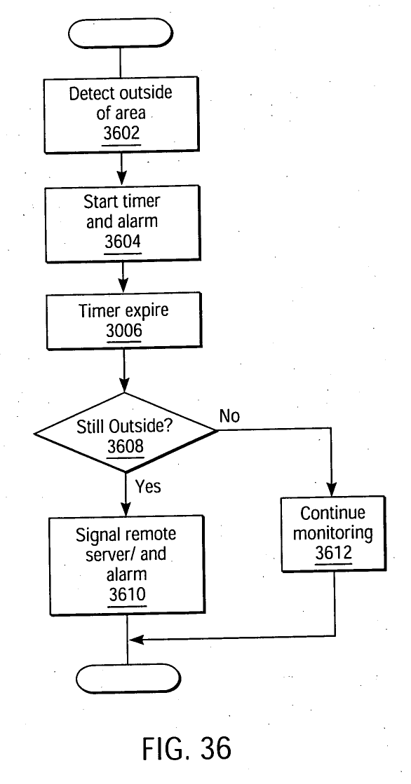

FIG. 12 depicts the nonlinear improvement of the net correlation shown in FIG. 11.

### DETAILED DESCRIPTION OF THE ILLUSTRATIVE EMBODIMENTS

By way of overview and explanation, the illustrative embodiments of the present invention depict a QRS complex detection technique in an ECG signal from a patient undergoing MRI. As a patient undergoes MRI, the magnet in the MRI machine causes blood flow-induced voltage potentials which interfere with the QRS complex component of the ECG signal. To minimize the effect of interference patterns on detection of the QRS complex, the present invention takes an ECG signal from a patient and uses this ECG signal to detect the shape in time unique to QRS complex in the patient undergoing analysis. Using the detected QRS complexes in the ECG signal for the patient undergoing analysis, a QRS complex template is derived. The QRS complex template has a shape in time representative of the combined shape in time for QRS complexes in the ECG signal of a particular patient.

Once the QRS complex template has been derived, the QRS complex is correlated with a continuous-in-time ECG signal from the patient. Correlation refers to a measure of the similarity of the two signals. When the QRS complex template is correlated with the continuous-in-time ECG signal, the present invention measures how similar the shape in time of the QRS complex template is to the shape in time of the continuous-in-time ECG signal during any given window of time. The window of time shifts from the beginning of the continuous-in-time signal until the end of the continuous-in-time signal.

The continuous-in-time ECG signal can be the same signal used to detect the unique shape in time for QRS complexes in this patient or, alternatively, the continuous-in-time ECG signal may be derived from a second ECG signal sample from the same patient. Next, a threshold is determined using the pre-recorded ECG signal. The threshold indicates that when exceeded, the shape in time of the continuous-in-time ECG signal correlates with the shape in time of the QRS complex template.

Finally, a real-time ECG signal from the same patient is received. The real-time ECG signal is correlated with the QRS complex template. By correlating the real-time ECG signal with the QRS complex template, the present invention measures how similar the shape in time of the ECG signal is to the QRS complex template. Should the correlation of the real-time ECG signal with the QRS complex template exceed the predetermined threshold, the MRI begins a predescribed data acquisition process.

Correlation function processing, as described herein, permits the use of more information about the time course (“shape”) of the QRS complex in the ECG signal (in one or multiple channels) than just the amplitude of the peak. Consequently, correlation function processing is more robust at deciphering QRS complex from interference than prior art QRS detection techniques. Furthermore, correlation function processing reduces the need for time consuming electrode repositioning. In addition, correlation function processing can be executed rapidly. In fact, the rapid computational speed of correlation function processing makes it competitive with simpler conventional amplitude thresholding approaches such as the voltage detection method discussed above. Consequently, correlation function processing introduces minimal delay in QRS trigger generation. Rather than triggering MRI upon the time of a voltage exceeding a threshold, correlation function processing triggers MRI upon the time of a correlation exceeding a threshold. In addition to the above described benefits, the present invention's use of correlation filtering better discriminates against interfering voltages, as interfering voltage will have a shape in time unlike the shape in time associated with QRS complex. Consequently, even if a interfering voltage has a similar amplitude voltage as QRS complex, a trigger will not be falsely generated because the shape in time of the interfering voltage will differ from QRS complex. The correlation function processing method of the present invention can be further improved through the use of a nonlinear processing step (as discussed below in FIG. 12). The nonlinear processing step further increases the conspicuity and detectability of QRS peaks.

In short, the correlation function processing of the present invention provides an improved new method to detect QRS complexes simply, in real time, and with minimal disruption by interfering voltages.

FIGS. 1-3 depict an exemplary embodiment of the QRS complex detection technique for a patient undergoing MRI in accordance with the exemplary embodiments of the present invention. More specifically, FIG. 2 depicts a flow diagram in accordance with a first exemplary method, while FIG. 3 depicts a flow diagram in accordance with a second exemplary method. The correlation filtering process proceeds in two stages as demonstrated in FIG. 5. First, a determination of a template for the shape of the QRS complex is made 570 from a received ECG signal. Second, this template 570 is correlated with the ECG in real time in order to detect QRS complexes while the patient undergoes MRI.

Beginning with FIG. 2, in step 212 a first ECG signal is received from a patient. The first ECG signal received can be recorded prior to the patient undergoing MRI, while the patient is in the MRI system magnet but prior to imaging, or finally while the patient is undergoing MRI. Detection of the QRS complex in the ECG signal can be accomplished by visual inspection of the recorded sample or through the use of other, more powerful, automated algorithms.

In step 214, the received ECG signal is recorded. While in FIGS. 2 and 3 the ECG signal is first received and then recorded, as one of ordinary skill in the art would appreciate, however a pre-recorded ECG signal could be used instead.

In step 216, each shape in time that is unique to the QRS complex is identified. Shape in time refers to a series of voltage values at discrete moments of time. Generally speaking, QRS complex has a unique shape. As shown in FIG. 4, the shape in time that is unique to QRS complex 450 comprises an R peak 468 which is a high peak value and two substantially lower peak values, Q and S peaks. Besides the QRS complex 450, also depicted in FIG. 4 are the P-, T-, and U-waves 462, 464, 466.

Just as QRS complex generally has shape in time that is unique to QRS complex, each QRS complex for a particular patient has a unique shape in time. The QRS complex shape will also vary depending on the location at which it is recorded from the surface of the body. QRS complex for a particular patient varies based on genetics as well as any malfunctions or heart conditions that may exist as discussed above. For instance, scar tissue in an individual patient will induce a QRS complex shape in time unique to a patient with such scar tissue. Once the shape in time that is unique to QRS complex has been identified in step 216, then a QRS complex template is determined in step 218.

The generation of a QRS template from a received ECG signal relies on the ability to use more powerful methods to detect representative QRS complexes, while eliminating the requirement of execution in real-time. For example, the generation of QRS template could include interactive “manual” identification of representative QRS complexes by visual inspection of the graphical display of the received ECG signal or a more automated method. For example, QRS complex could be detected with an automated method, such as looking for the occurrence of voltage peaks or peaks in the time derivative of the voltage. These detections could be considered initial “candidate” QRS complexes. Interactively these “candidate” QRS complexes could be culled to eliminate false detections. Alternatively, assuming that most “candidate” QRS complexes are correct QRS detections, we can use these initial candidates to generate an initial QRS template that should be close to correct. This can then be correlated (as described below) with the individual QRS candidates initially detected, in order to more automatically reject false detections. The remaining QRS complexes can then be used to generate a final template.

In step 218, the QRS complex template is determined by examining each shape in time for each QRS complex identified in the ECG signal, and calculating a QRS complex whose shape in time represents the average shape in time of the previous identified QRS complexes. In other words, the QRS complex template represents a representative example of all the QRS complexes identified for this particular patient. In selecting the segment of the QRS complex for use in the QRS complex template, the portion leading up to the peak QRS amplitude (positive or negative) is preferred. When the QRS complex template is finally correlated with a real-time ECG signal, if the portion leading up to the peak QRS amplitude is used, there will be minimal delay in trigger generation when the peak of the QRS complex is detected.

The template generation process is further schematically illustrated in FIG. 6. As shown in FIG. 6, first the ECG samples are recorded 614. Second, QRS complexes are detected 616. Third, the selected QRS complexes are averaged to find the QRS template 618. The template generation itself can be carried out by simply temporally registering the QRS complexes detected in the recorded ECG and then averaging them point by corresponding point.

Returning again to FIG. 2, in step 222 the QRS complex template, which is a single QRS complex shape, is correlated with a continuous-in-time ECG signal sample having multiple QRS complexes. It should be noted that the continuous-in-time ECG signal could, in fact, be a prior received ECG signal in which the unique shapes in time for QRS complex were initially identified. Alternatively, a new ECG signal sample that is continuous-in-time could be provided and/or received and recorded for use in the correlation function in step 222.

The process of correlating the ECG signal can be accomplished in a number of ways. While one such way will be explained below for exemplary purposes, those of skill in the art will recognize that the herein explained correlating process is just one of many possible correlating processes. For purposes of illustration, the process of correlating the ECG signal, S(t), digitized at a set of discrete time points, with a discrete set of template values, T(t′), is carried out by multiplying the two point-by-corresponding-point for sampled times and then summing the results, for each consecutive temporal offset of the template:

$$R ( t ) = S ( t ) * T ( {t}^{′} ) = {\sum }_{i = 1}^{N} S [ t - ( N - i ) Δ t ) ] T ( i ) \qquad ( 1 )$$

where R(t) is the output of the correlation process, Δt is the temporal sampling interval of the ECG signal, and there are N time points in the QRS template. In testing candidate QRS complexes, only the temporal offset corresponding to the best alignment of the template and the candidate complex must be calculated. The same correlation process can be carried out on the ECG signal as it is detected in real time, using the N most recently detected values. This process is equivalent to using a tapped delay line on the detected signal, with weights for the successive delays determined by the template.

FIG. 7 best depicts the correlation process just described. FIG. 7 depicts three channels with three corresponding ECG signals, namely S₁(t), S₂(t), and S₃(t), each depicting three QRS complexes. Equation (1) mathematically describes the process of correlating the three ECG signals with the QRS template, prior to calculating the net correlation 729, which will be described below with reference to the second embodiment of the present invention. As shown in FIG. 7, the correlation processing function generates a QRS template R₁(t), R₂(t), and R₃(t) for each of the ECG signals S₁(t), S₂(t), and S₃(t).

In step 224, using the continuous-in-time ECG signal and the calculated QRS complex template, a threshold is determined. The threshold represents a correlation value that when exceeded indicates that the continuous-in-time ECG signal closely correlates with the QRS complex template. For example, as the QRS complex template is compared with the continuous-in-time ECG signal sample in step 222, it will be determined that at times where the QRS complex template does not correlate with the ECG signal, the value will be low compared to the threshold. Alternatively, at times when the correlated QRS complex template is correlated with the continuous-in-time ECG signal, the correlation value will be high compared to the threshold.

To estimate the threshold or in other words the magnitude of the peak correlation (“signal”) to be expected when detecting a QRS complex, we can find the peak correlations (no temporal offset) of the template with each of the originally detected QRS complexes from the recorded ECG (which were used to create the template) and find their mean value. The results can also be used to estimate the range or standard deviation of peak values to be expected in QRS detection. Similarly, correlation of the template with the recorded ECG between the QRS complexes can be used to estimate the peak background correlation values (“noise”) to be expected. This can, in turn, be used to estimate an initial threshold (above the noise peaks but below the true QRS peaks) for the output of the correlation process, to be used in subsequent real-time detection of the QRS complexes.

In step 226, a real-time ECG signal is received from the patient undergoing MRI. At this point, in step 232 the patient is lying in the MRI machine with ECG probes attached to an ECG machine. The ECG signal is then correlated with the QRS complex template in step 228. Execution of the QRS detection process in real time is carried out by the ongoing correlation of the ECG signal with the template derived as described mathematically above in Equation 1 and pictorially by FIG. 7, in part. The output of the correlation is compared with a threshold, and a trigger pulse can be generated when it exceeds the threshold. The threshold can be interactively adjusted as necessary for reliable detection of the QRS complexes.

In step 232, it is determined whether the correlated real-time ECG signal from the patient undergoing MRI has exceeded the predetermined threshold. Should, in step 232, the threshold be exceeded, then the MRI initiates a predescribed MRI data acquisition function in step 234. On the other hand, if the threshold has not yet been exceeded by the real-time ECG signal while the patient undergoes MRI, then the system continues to receive real-time ECG signals in step 226 until the threshold has been exceeded.

When the real-time ECG signal exceeds a threshold, a trigger is sent to the MRI machine to initiate a type of data acquisition. By way of example, the system could send a 5 volt internal or external trigger input to the MRI machine. Once the MRI machine receives the trigger input, the MRI machine initiates the data acquisition.

While the second embodiment of the present invention in FIG. 3 generally follows the steps of first embodiment, some additional steps are involved. FIG. 3 represents a multi-channel, multi-ECG signal arrangement. While in this case a QRS complex template is determined in step 318, this QRS complex template represents a QRS complex that is representative of the shape in time that is unique to QRS complex in ECG signals originating from multiple ECG channels. Accordingly before the QRS complex template is determined, the system must identify in step 317, a common window of time in which generally QRS complex occurs in the multiple channels.

A QRS complex template is derived from the signals occurring during this same window of time for multiple channels. If the window of time is not chosen, then the present invention would not know from which window of time to derive the QRS complex template. By way of analogy, should a person have two watches, each having a different time, that person will never know which of the watches has the correct time. Similarly, if the present invention does not pick a single window of time, the present invention will not know when the QRS complex is occurring so as to determine a representative QRS complex template for each of the multiple channels. Generally, the peak magnitude voltage of the QRS complex will not be the same in each channel. Accordingly, the time of the temporal segments or in other words the window of time used for the QRS complex template should match in each channel. The window of time chosen is selected in accordance with the channel with the “best” QRS complexes. By way of example, should channel one depict the clearest or in other words the best QRS complexes, the window of time for generation of the QRS complex templates for the remaining channels will occur at the same window of time as channel one. As described above, generally it is preferred that the segment of QRS complex taken from the ECG signal in channel one during this window of time would be the portion leading up to the peak amplitude of QRS complex in an effort to reduce triggering delays associated with MRI.

In addition to picking a window of time, another difference between the single and multichannel embodiments is that being multiple channels are involved a weighted score must be determined in step 323 for each channel. The weighted score is described below with reference to equations (2-3). If a particular channel has a stronger QRS complex than another channel, that channel will be assigned an overall higher weighted score. This weighted score determined in step 323 is then used in determining the threshold for the combined channels. For example, a strong channel will have an ultimately heavier contribution to the threshold than an channel with a weak QRS complex.

Therefore in step 329, once the patient undergoes MRI, the correlation of the real-time ECG signal with QRS complex will be combined proportionate to the weighted score for that particular channel. In so doing, the system ensures that if a weak channel closely correlates with the QRS complex template that the weak channel does not supercede a distinct correlation of QRS complex template that a strong channel has detected.

To use this method with simultaneous monitoring of multiple ECG channels, Sₖ(t), where k is the channel number, a sample temporal segment of the multiple channel signals is recorded. FIG. 7 depicts the multichannel approach. This time, the ECG channel with the most prominent QRS complexes is used to identify candidate QRS complex events and a QRS template for the channel is interactively created as above, with culling of any false detections. For purposes of illustration, if channel 1 in FIG. 7 had the most prominent QRS complexes, the correlated QRS template of channel 1, namely, R₁(t), would receive a higher score a₁(t) indicating that channel should be more heavily relied upon for the net correlation 729. The corresponding (temporally registered) recorded segments of the other ECG channels are then used to generate corresponding QRS templates for each channel, Tₖ(t′). A signal-to-noise ratio, SNRₖ, can be estimated for each channel, as described above.

A net correlation value can then be formed from a weighted combination of the results of correlating each channel with its corresponding template as defined below:

$$R ( t ) = {\sum }_{k} {a}_{k} {S}_{k} ( t ) * {T}_{k} ( {t}^{′} ) . \qquad ( 2 )$$

The values of the channel weights, aₖ, can be chosen according to the relative SNR values for each channel. For example, we can use:

$$\begin{matrix}{a}_{k} = {SNR}_{k} - 1 , {SNR}_{k} \geq 1 \\ = 0 , {SNR}_{k} < 1\end{matrix} \qquad ( 3 )$$

FIG. 7 depicts the net correlation 729 of the three ECG signals, S₁(t), S₂(t), and S₃(t), from the three ECG channels. As described above, if one channel has more prominent QRS complexes over another channel, such channel's correlation will more heavily influence the net correlation 729.

While the process of assigning a net correlation value can be formed through the use of the process described with equations (2) and (3), as those of ordinary skill in the art will appreciate, other processes can be used. The present invention is not confined to the process of assigning a net correlation in accordance with equations (2) and (3), but instead equations (2) and (3) are for purposes of illustration.

By way of example, FIGS. 8A through 12 depict the correlation function processing method of the present invention post the initial QRS complex template determination which is depicted in FIG. 7. First, FIGS. 8A and 8B demonstrate the problem associated with identifying QRS complex while the patient is in the MRI machine. FIG. 8A depicts a three-channel ECG signal recorded outside the MRI magnet, while FIG. 8B depicts the three-channel ECG signals inside the MRI magnet. Clearly, the QRS complexes 800 are easier to decipher from the ECG signals in FIGS. 8A than 8B. Accordingly, FIG. 8B demonstrates the ability of the magnet in the MRI machine to cause interfering voltages. Second, the corresponding QRS templates for each channel shown in FIG. 8B are shown in FIG. 9. As seen in FIG. 9, each QRS template for each channel differs significantly from its neighboring channel. The results of correlating each channel shown in FIG. 8B with its template are shown in FIG. 10. The benefit of the correlation of each channel in FIG. 8B with its corresponding QRS template in FIG. 9 is demonstrated by a comparison of the original signals shown in FIG. 8B and the correlation signals in FIG. 10. FIG. 10 depicts three-channels with more clearly distinctive QRS complexes than shown in FIG. 8B. At this point in time, a net correlation 729 has not been performed. That being said, the net correlation 729 of the channels (weighted by their respective SNRs) correlated in FIG. 10 is shown in FIG. 11, along with the corresponding original ECG signals and the triggers generated from the net correlation by a comparison threshold. The net correlation 1129 even more clearly depicts the QRS complexes found in the ECG signal of the patient. FIG. 11 depicts stacked one on top of the other the three original ECG signals and the net correlation ECG signal 1129. Each ECG channel contribution is weighted, as discussed above, by how prominently the QRS complex was displayed in each ECG signal. Also depicted in FIG. 11 is the triggering of the MRI. Note that the MRI is triggered upon detection of QRS complex. An improved version of the FIG. 11 is demonstrated in FIG. 12. FIG. 12 demonstrates a nonlinear processing set which ultimately more clearly depicts QRS complex in the resulting net correlation 1129 shown in FIG. 11. Essentially, FIG. 12 depicts the corresponding results of taking the cube of the output of the correlation filter as shown in FIG. 11. As long as the peaks of the correlation-filtered signal are sufficiently above the background noise, the conspicuity of the peaks can further be increased by using a nonlinear processing step. Accordingly, FIG. 12 demonstrates the process of taking the cube of the processed signal. A comparison of the net correlation 1229 in FIG. 12 with the net correlation 1129 in FIG. 11 reveals that FIG. 12 has further eliminated interfering voltages through the use of the nonlinear step.

FIG. 1 depicts the system overview of the present invention. As shown in FIG. 1, first the patient is attached to the ECG without the presence of the MRI 80. An ECG signal 60 is then displayed to a technician. From the ECG display, the QRS complexes 50a, 50b are discerned from the ECG signal. As shown in FIG. 1, the QRS complex has a shape in time that is unique to QRS complex. As demonstrated, besides ECG signal other interference patterns 52a, 52b are present in the ECG signal, some of which exceed the voltage potential of the QRS complex 52b. The QRS complex processor, which could exist externally or alternatively internally in an MRI system, then devises a QRS complex template 55 for the patient. The QRS complex template represents the shape in time that is unique to QRS complex for this particular patient.

Next, the QRS complex template 55 is correlated with a continuous in time ECG signal 60 from the same patient. As shown the QRS complex template 55 is compared with the continuous in time ECG signal 60 to determine how closely the signals correlate. Essentially, the QRS complex template is superimposed on the continuous in time ECG signal. In this figure, the continuous in time signal is the same as the original signal sample, namely 60, however a new continuous in time signal can also be correlated as long as the new continuous in time signal is derived from the same patient.

As the QRS processor correlates the QRS complex template 55 with the continuous-in-time ECG signal 60, the processor recognizes a threshold. The value of the correlation relative to the threshold indicates that at this window in time the continuous-in-time ECG signal either does or does not closely correlate with the QRS complex template. The window of time, e.g. a 15 milliseconds window of time, shifts from the beginning of the continuous-in-time signal to the end of the continuous-in-time signal. In this manner, should the QRS complex template be compared against a window of time in the continuous-in-time ECG signal that comprises mostly interference, the correlation will be low. However, when the QRS complex template is compared against a window of time in the continuous-in-time ECG signal that comprises a QRS complex, the correlation will be high. In a preferred embodiment a high value indicates a close correlation; the opposite could also give the same indication. For example a low threshold value could indicate a close correlation, while a high threshold value could indicate a disparate correlation.

While in FIG. 1, two ECG systems are shown, the same ECG machine could also be used. Same applies to the MRI machines. While in FIG. 1, two MRI machines are shown, the same MRI machine could also be used. Now the QRS complex template 55 is correlated with a real-time ECG signal. Each time the threshold is exceeded, a trigger pulse is sent by the trigger 85 to the MRI 80 to indicate that the MRI should begin a particular kind of data acquisition or alternatively should update a type of data acquisition.

It should be noted that in a preferred embodiment, the ECG signal is digitized (for example at 250 Hz) and recorded on a workstation, laptop, or other PC computer with a commercial interface program such as, but not limited the LabView software and or hardware manufactured by National Instruments. A number of ECG channels and respiratory channels can be recorded simultaneously. Once recorded, the ECG is preprocessed with digital bandpass filtering to reduce the effects of baseline variation, line frequency interference and flow-induced potentials. After the ECG has been preprocessed, a sample of the ECG is saved (such as, for example, a 12 second duration) and templates corresponding to the Q wave peaks are interactively selected from the templates for each channel. Correlation of the ECG channels with their corresponding templates is then carried out in real-time, and thresholding of the combined correlation functions is used to detect the QRS peaks. These peak detections are then supplied as external trigger inputs to the MRI system.

In addition, while the electrocardiogram has been described using the abbreviation ECG, as one of skill in the art will appreciate, the abbreviation EKG could also have been chosen.

In addition, it should be noted that the ECG signal comprises QRS complex as well as a number of other signal components. QRS complex corresponds to the initiation of the heart beat in a patient. More specifically, QRS complex corresponds to the contraction of the ventricles of the heart. While the present invention has been described with reference to detecting a QRS complex, the system is equally applicable to the detection of other signal components of an ECG signal, such as the P- and T-wave of the ECG signal. The P-wave corresponds to the contraction of the atria, while the T-wave corresponds to the relaxation of the ventricles.

The above method was implemented with a laboratory-built 3-channel ECG amplifier, using pair-wise combinations of signals from four electrodes placed over the precordium; a fifth lead was placed near the left shoulder for common mode rejection. The signals were amplified and digitized at 250 Hz for each channel, using a digitizer (DAQ card 1200, National Instruments) under control of a custom-written LabVIEW program (National Instruments), running on an 800 MHz laptop PC (KDS, Valiant 600 Series, 128 Mb RAM). A second order Butterworth bandpass filter was applied to the data, with lower limit of 2.5 Hz and upper limit of 50 Hz. A graphical user interface (GUI) was used for controlling the execution of the LabVIEW program; the program also carried out the QRS template creation and real time correlation for QRS detection and trigger generation as described above. A 12 second segment of the ECG was recorded for generation of the templates and 16 time points (64 ms) were included in each template. The program provides real time displays of the separate channel signals, as well as of the results of the template correlation and the peak detections. In operation, after visual inspection of the incoming signals to verify correct ECG signal detection, the operator can initiate the peak detection process with a single button press that starts automatic recording of the calibration segment recording, generation of the templates, calculation of the correlation weights and initial correlation threshold, and then proceeds to real-time peak detection. The detection process parameters can then be interactively adjusted, if necessary.

Thus, while there have been shown, described, and pointed out fundamental novel features of the invention as applied to a preferred embodiment, it will be understood that various omissions, substitutions, and changes in the form and details of the devices illustrated, and in their operation, may be made by those skilled in the art without departing from the spirit and scope of the invention. For example, it is expressly intended that all combinations of those elements steps which perform substantially the same, function in substantially the same way, to achieve the same results are within the scope of the invention. Substitutions of elements from one described embodiment to another are also fully intended and contemplated. It is also to be understood that the drawings are not necessarily drawn to scale, but that they are merely conceptual in nature. It is the intention, therefore, to be limited only as indicated by the scope of the claims appended hereto.

What is claimed is:

1. Method for automating an initiation of MRI data acquisition upon detection of QRS complex in an ECG signal for a patient undergoing MRI, comprising the steps of:
correlating a QRS complex template with a continuous-in-time ECG signal of a patient, the QRS complex template representative of a shape in time unique to QRS complex in a set of QRS complexes for the patient;
determining a threshold that when exceeded indicates that the continuous-in-time ECG signal substantially correlates with the QRS complex template;
correlating a real-time ECG signal of the patient while undergoing MRI with the QRS complex template; and,
initiating automatically a prescribed MRI data acquisition at a point in time when the correlation of the real-time ECG signal with the QRS complex template exceeds the threshold.

2. Method as in claim 1, further comprising:
receiving the real-time ECG signal from an ECG test probe attached at one end to the patient and at the other end to an ECG machine.

3. Method as in claim 1, further comprising:
indicating on a display of voltage versus time, the shape in time unique to the QRS complex in said set of QRS complexes in the patient, the shape in time comprising a Q peak, an R peak, and an S peak of the QRS complex.

4. Method as in claim 1, wherein the first correlating step further comprises:
superimposing the QRS complex template over the continuous-in-time ECG signal.

5. Method as in claim 4, wherein the superimposing step further comprises:
continuously shifting forward in time the superimposed QRS complex template over the continuous-in-time ECG signal.

6. Method as in claim 1, wherein the determining step further comprises:
assigning a high correlation value when during a particular window of time the QRS complex template substantially correlates with the continuous-in-time ECG signal; and
assigning a low correlation value when during a particular window of time there is an absence of a substantially close correlation of the QRS complex template with the continuous-in-time ECG signal.

7. A method as in claim 6, wherein the determining step further comprises:
continuously shifting forward in time the window of time.

8. A method as in claim 1, wherein the second correlating step further comprises:
superimposing the QRS complex template over the real-time ECG signal from the patient undergoing MRI.

9. A method as in claim 8, wherein the superimposing step further comprises:
continuously shifting forward in time the superimposed QRS complex template over the real-time ECG signal.

10. A method as in claim 1, wherein prior to the initiating step a MRI machine receives a trigger pulse indicating an initiation of a prescribed MRI data acquisition.

11. A method as in claim 1, wherein the prescribed MRI data acquisition comprises at least one of:
updating a type of data being acquired and, initiating an initiation of a data acquisition process.

12. Method for automating an initiation of MRI data acquisition upon a detection of QRS complex in an ECG signal for a patient undergoing MRI, comprising the steps of:
correlating a QRS complex template with each continuous-in-time ECG signal received from a set of ECG channels of a patient, the QRS complex template representative of a shape in time unique to a QRS complex in a set of QRS complexes for the patient;
assigning a weighted score for each ECG channel indicative of a strength of the correlation of the QRS complex template with the continuous-in-time ECG signal for a particular ECG channel in said set of ECG channels;
determining a threshold that when exceeded indicates that the continuous-in-time ECG signal correlates with the QRS complex template, the threshold a combined value of each continuous-in-time ECG signal in said set of ECG channels, the contribution of each ECG channel to the threshold proportionate to the assigned weighted score for each ECG channel;
correlating the QRS complex template for each ECG channel in said set of ECG channels with a real-time ECG signal for each ECG channel in said set of ECG channels of the patient undergoing MRI;
combining the correlations for each ECG channel in said set of ECG channels, the contribution of each ECG channel to the combined correlation proportionate to the weighted score assigned to each ECG channel; and,
initiating automatically a prescribed MRI data acquisition at a point in time when the combined correlation exceeds the threshold.

13. A method as in claim 12, wherein the first correlating step further comprises:
choosing a window of time for the correlation of QRS template with the continuous-in-time ECG signal in a single ECG channel that is representative of a window of time at which QRS complex generally occurs in the remaining ECG channels.

14. A method as in claim 12, wherein the assigning step further comprises:
associating a higher weighted score for an ECG channel having a stronger correlation of the QRS complex template with the continuous-in-time ECG signal; and
associating a lower weighted score for an ECG channel having a weaker correlation of the QRS complex template with the continuous-in-time ECG signal.

15. A method as in claim 12, wherein the threshold comprises an overall threshold for each ECG channel, individual thresholds contributing to the overall threshold proportionate to the weighted score associated with each ECG channel.

16. A method for automating an initiation of MRI data acquisition upon detection of QRS complex in an ECG signal for a patient undergoing MRI, comprising the steps of:
determining a QRS complex template having a shape in time representative of an average shape in time of QRS complex in a set of QRS complexes in an ECG signal for a patient;
correlating the QRS complex template with a continuous-in-time ECG signal of the patient;
determining a threshold that when exceeded indicates that the continuous-in-time ECG signal correlates with the QRS complex template;
correlating a real-time ECG signal from the patient undergoing MRI with the QRS complex template; and,
initiating automatically a prescribed MRI data acquisition at a point in time when the correlation of the real-time ECG signal with the QRS complex template exceeds the threshold.

17. Method as in claim 16, wherein the first correlating step further comprises:
superimposing the QRS complex template over the continuous-in-time ECG signal sample.

18. Method as in claim 17, wherein the superimposing step further comprises:
continuously shifting forward in time the superimposed QRS complex template over the continuous-in-time ECG signal sample.

19. Method as in claim 16, wherein the second determining step further comprises:
assigning a high correlation value when during a particular window of time the QRS complex template substantially correlates with the continuous-in-time ECG signal; and
assigning a low correlation value when during a particular window of time there is an absence of a substantially close correlation of the QRS complex template with the continuous-in-time ECG signal.

20. A method as in claim 19, wherein the window of time continuously shifts.

21. A method as in claim 16, wherein the second correlating step further comprises:
superimposing the QRS complex template over the real-time ECG signal of the patient undergoing MRI.

22. A method as in claim 21, wherein the superimposing step further comprises:
continuously shifting forward in time the superimposed QRS complex template over the real-time ECG signal.

23. A method as in claim 16, further comprising prior to the initiating step receiving a trigger pulse which indicates a time at which to initiate the initiation of the prescribed MRI data acquisition.

24. A method as in claim 16, wherein the prescribed MRI data acquisition comprises at least one of an update of a type of data being acquired and an initiation of a data acquisition process.

25. A computer system for detecting a QRS complex, the computer system comprising:
a memory; and
a processor interconnected with the memory and having at least one software component loaded therein,
wherein the software component causes the processor to execute the steps of method according to claim 1.

26. A computer program product comprising a computer readable medium having a software component encoded thereon in computer readable form, wherein the software component may be loaded into a memory of a computer system and cause a processor interconnected with the memory to execute the steps of a method according to claim 1.

27. Method for automating an initiation of MRI data acquisition upon detection of QRS complex in an ECG signal for a patient undergoing MRI, comprising the steps of:
correlating a QRS complex template with a continuous-in-time ECG signal of a patient, the QRS complex template representative of a shape in time unique to QRS complex in a set of QRS complexes for the patient;
determining a threshold that when exceeded indicates that the continuous-in-time ECG signal substantially correlates with the QRS complex template;
correlating a real-time ECG signal of the patient while undergoing MRI with the QRS complex template

28. Method for automating an initiation of MRI data acquisition upon correlation of a real-time ECG signal of a patient undergoing MRI with a predescribed template, comprising the steps of:
correlating a predescribed template with a continuous-in-time ECG signal of a patient, the predescribed template representative of a time course unique to a subsection of the ECG signal for the patient in a series of subsections of the ECG signal for the patient;
determining a threshold that when exceeded indicates that the continuous-in-time ECG signal substantially correlates with the predescribed template;
correlating a real-time ECG signal of the patient while undergoing MRI with the predescribed template.

29. A method as in claim 28, further comprising:
initiating automatically a prescribed MRI data acquisition when the correlation of the real-time ECG signal with the predescribed template exceeds the threshold.

30. A method as in claim 28, further comprising:
determining the time course unique to the subsection of the ECG signal from a visual display of the ECG signal.

31. A method as in claim 28, further comprising, wherein the first correlating step further comprises:
superimposing the predescribed template over the continuous-in-time ECG signal.

32. A method as in claim 31, wherein the superimposing step further comprises:
continuously shifting forward in time the superimposed predescribed template over the continuous-in-time ECG signal.

33. A method as in claim 28, wherein the determining step further comprises:
assigning a high correlation value when during a particular temporal segment the predescribed template substantially correlates with the continuous-in-time ECG signal; and
assigning a low correlation value when during a particular temporal segment there is an absence of a substantially close correlation of the predescribed template with the continuous-in-time ECG signal.

34. A method as in claim 28, wherein the second correlating step further comprises:
superimposing the predescribed template over the real-time ECG signal from the patient undergoing MRI.

35. A method as in claim 34, wherein the superimposing step further comprises:
continuously shifting forward in time the superimposed predescribed template over the real-time ECG signal.

36. Method for automating an initiation of MRI data acquisition upon correlation of a real-time ECG signal of a patient undergoing MRI with a predescribed template, comprising the steps of:
correlating a predescribed template with each continuous-in-time ECG signal received from a set of ECG channels of a patient, the predescribed template representative of a time course unique to a subsection of the continuous-in-time ECG signal for the patient;
assigning a weighted score for each ECG channel indicative of a strength of the correlation of the predescribed template with the continuous-in-time ECG signal for a particular ECG channel in said set of ECG channels;
determining a threshold that when exceeded indicates that the continuous-in-time ECG signal correlates with the predescribed template, the threshold a combined value for each continuous-in-time ECG signal in said set of ECG channels, the contribution of each ECG channel to the threshold proportionate to a weighted score assigned to each ECG channel;
correlating the predescribed template for each ECG channel in said set of ECG channels with a real-time ECG signal for each ECG channel in said set of ECG channels of the patient undergoing MRI;
combining the correlations for each ECG channel in said set of ECG channels, the contribution of each ECG channel to the combined correlation proportionate to the weighted score assigned to each ECG channel; and,
initiating automatically a prescribed MRI data acquisition when the combined correlation exceeds the threshold.

37. A method as in claim 36, wherein the first correlating step further comprises:
choosing a temporal segment for the correlation of predescribed template with the continuous-in-time ECG signal in a single ECG channel which clearly depicts the time course unique to the subsection of the continuous-in-time ECG signal.

38. A method as in claim 36, wherein the assigning step further comprises:
associating a higher weighted score for an ECG channel having a stronger correlation of the predescribed template with the continuous-in-time ECG signal; and
associating a lower weighted score for an ECG channel having a weaker correlation of the predescribed template with the continuous-in-time ECG signal.

39. A method as in claim 38, wherein the threshold comprises an overall threshold for each ECG channel, individual thresholds of each ECG channel contributing to the overall threshold proportionate to the weighted score associated with each ECG channel.
